(19) United States
(12) Patent Application Publication
(10) Pub. No.: US 2025/0017753 A1
(43) Pub. Date: Jan. 16, 2025

(54) ELBOW ORTHOSIS AND PROCESS FOR ADAPTING A CONFIGURATION OF SUCH AN ORTHOSIS TO A PATIENT

(71) Applicants: INSTITUT NATIONAL DE LA SANTE ET DE LA RECHERCHE MEDICALE, Paris (FR); ASSISTANCE PUBLIQUE - HOPITAUX DE PARIS, Paris 12 (FR); UNIVERSITÉ VERSAILLES SAINT-QUENTIN-EN-YVELINES, Versailles (FR); LEXAT, Gargenville (FR)

(72) Inventors: Jean-Paul CARTA, Gargenville (FR); Sébastien CHARLES, Velizy Villacoublay (FR); Gilbert PRADEL, Bourg-La-Reine (FR); Frédéric BARBOT, Garches (FR)

(21) Appl. No.: 18/704,239

(22) PCT Filed: Oct. 28, 2022
(86) PCT No.: PCT/EP2022/080205

§ 371 Date: Apr. 24, 2024

(30) Foreign Application Priority Data
Oct. 29, 2021 (EP) 21306527.9

(51) Int. Cl. A61F 5/01

(52) U.S. Cl. CPC A61F 5/013; A61F 2005/0155; A61F 2005/0167

(57) ABSTRACT

This elbow orthosis comprises a first fitting, a second fitting and a mechanical stop arrangement (40) configured for limiting the angular movement of the second fitting with respect to the first fitting around a first rotation axis (A1). The mechanical stop arrangement (40) includes first and second plates (402, 404), each provided with a circular slot (4024, 4044) and a series of through holes (4028, 4048) spread on a circle centered on a central axis (A402, A404) of the circular, and a crank (408) equipped with a pin (409) engaged in the circular slots (4024, 4044). The first and second plates (402, 404) are aligned on a common central axis and their relative orientation around this common axis is set by aligning at least one through hole (4028) of the first plate with one through hole (4048) of the second plate. Some portions of the circular slots (4024, 4044) overlap, depending on the orientation of the first and second plates around the common axis. The extremities (4024A, 4024B, 4044A, 4044B) of the overlapping portions of the circular slots define the amplitude of the maximum angular movement of the second fitting with respect to the first fitting when it is driven by a worm-and-wheel transmission (22).

The present invention concerns an elbow orthosis, which can be used for the rehabilitation of the arm of patients affected by spasticity, for instance after a cardio-vascular accident or CVA.

One issue with these patients is that they cannot fully control their muscles, so that re-education must start with repeated movements of flexion/extension of the elbow.

In this context, several elbow-orthoses are known for moving the forearm of a patient with respect to his/her arm, thanks to two fittings articulated together and respectively mounted around the arm and around the forearm of the patient.

For instance, the Active Elbow Orthosis mentioned in the article of Riple et al published in the International Journal of Advanced Robotic Systems (2014, 11:143) includes two fittings respectively configured to be mounted on an arm and on a forearm of a patient, an electric actuator and a worm-and-wheel transmission for transmitting the output movement of the actuator to a fitting attached to the patient's forearm. This orthosis includes a safety mechanical stop arrangement for limiting the angular relative movement between the two fittings. This safety mechanical stop arrangement includes two aluminum switches whose position must be set in advance, which is cumbersome and not intuitive. In addition, this mechanism is complex, thus relatively expensive. The adaptation of the configuration of the orthosis to its actual use, in particular for a left or right arm, takes time and requires a highly qualified operator.

The present invention aims at solving these problems by providing a new elbow orthosis with an improved mechanical stop arrangement, which facilitates the setting of the orthosis for the angular relative movement between the first and second fittings of the orthosis.

To this end, the present invention relates to an elbow orthosis comprising a first fitting configured to be mounted on an arm of a patient, a second fitting configured for being mounted on a forearm of the patient, an articulation mechanism between the first and second fittings and an electric gear-motor mounted on the first fitting for moving the second fitting with respect to the first fitting in rotation around a first rotation axis of the articulation mechanism. An output shaft of the electric gear-motor is aligned on a second rotation axis perpendicular to the first rotation axis. The orthosis is equipped with an angular sensor for sensing the angular position of the second fitting with respect to the first fitting around the first rotation axis and a torque sensor for sensing a torque exerted on the articulation mechanism by the electric gear-motor. A worm-and-wheel transmission drives a rotation shaft, which defines the first rotation axis, from the rotational movement of the output shaft of the electric gear-motor. The second fitting is secured to the rotation shaft. A mechanical stop arrangement is configured for limiting the angular movement of the second fitting with respect to the first fitting around the first rotation axis. According to the invention,
the mechanical stop arrangement includes
   a first plate provided with a first circular slot and a first series of through holes spread on a circle centered on a central axis of the first circular slot;
   a second plate provided with a second circular slot and a second series of through holes spread on a circle centered on a central axis of the second circular slot; and
   a crank secured to the rotation shaft and equipped with a pin engaged in the first and second circular slots;
the first and second plates are aligned on a common central axis and their relative orientation around this common axis is set by aligning at least one through hole of the first series with one through hole of the second series;
some portions of the first and second circular slots overlap, depending on the orientation of the first and second plates around the common axis; and
the respective extremities of the overlapping portions of the first and second circular slots define the amplitude of the maximum angular movement of the second fitting with respect to the first fitting, around the first rotation axis, when it is driven by the worm-and-wheel transmission.

Owing to the invention, the elbow orthosis may be easily adapted to the morphology and pathology of a patient, in particular for what concerns the end positions of the angular movements between the first and second fittings and the choice between his/her left and right arms.

According to advantageous and non-compulsory aspects of the invention, the elbow orthosis of the invention might incorporate the features of any one of claims 2 to 11.

According to another aspect, the present invention relates to a process for adapting a configuration of an elbow orthosis of the type mentioned here-above to the morphology and/or pathology of a patient, this method including at least the following steps:
a) adjusting the respective angular position of the first and second plates around their common central axis, so as to define with their overlapping first and second circular slots a path for the pin mounted on the crank secured to the rotation shaft; and
b) immobilizing the first and second plates in their respective angular positions by insertion of a locking member in one through hole of the first series and in one through hole of the second series.

According to advantageous and non-compulsory aspects of the invention, this process might incorporate the features of any one of claims 13 to 15.

The invention will be better understood on the basis of the following description of an elbow orthosis according to the invention, given as an illustrative example only, in reference to the annexed drawings where:

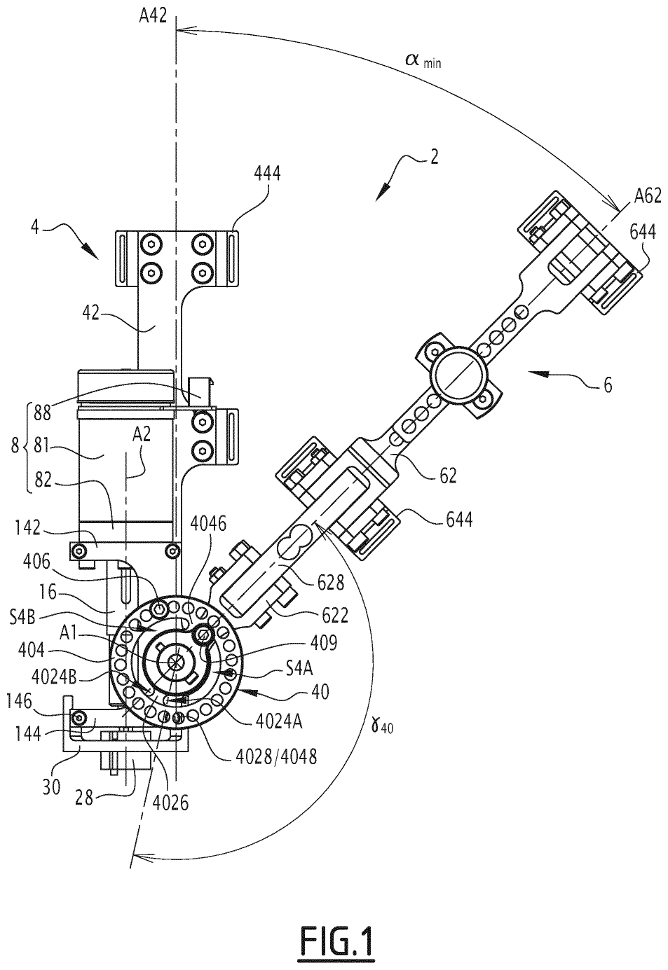

FIG. 1 is a side view of an orthosis according to the invention configured for use with a right arm of a patient and represented in a flexed position;

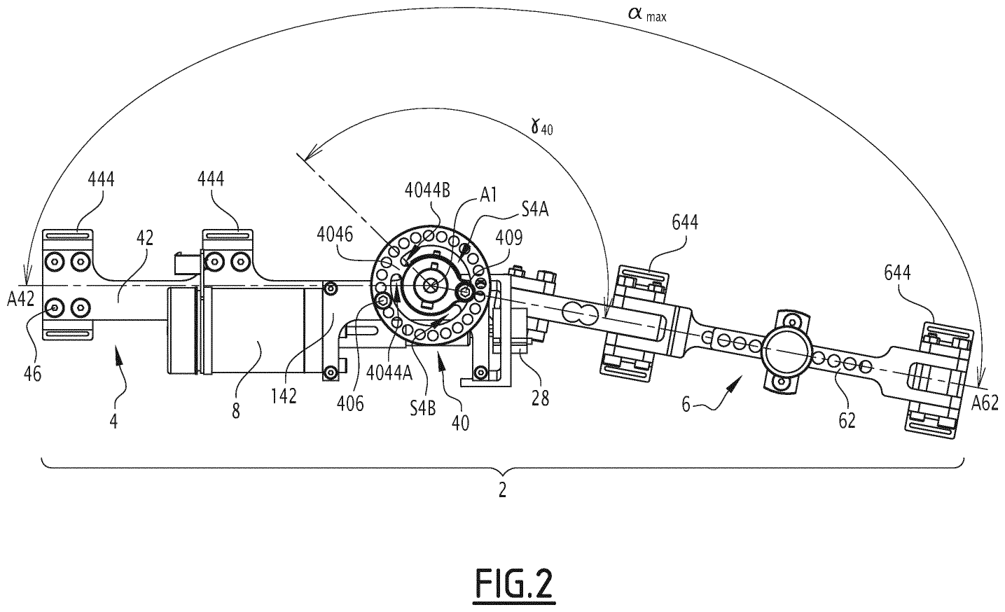

FIG. 2 is a side view similar to FIG. 1 when the orthosis is in an extended position;

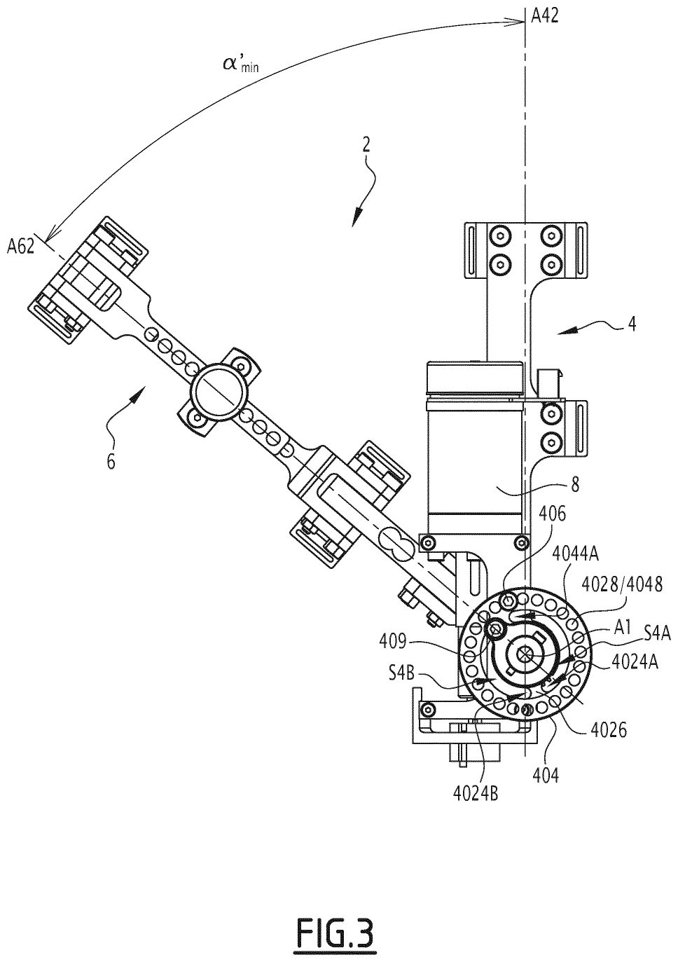

FIG. 3 is a side view comparable to FIG. 1 when the orthosis is configured for use on the left arm of a patient and represented in a flexed position;

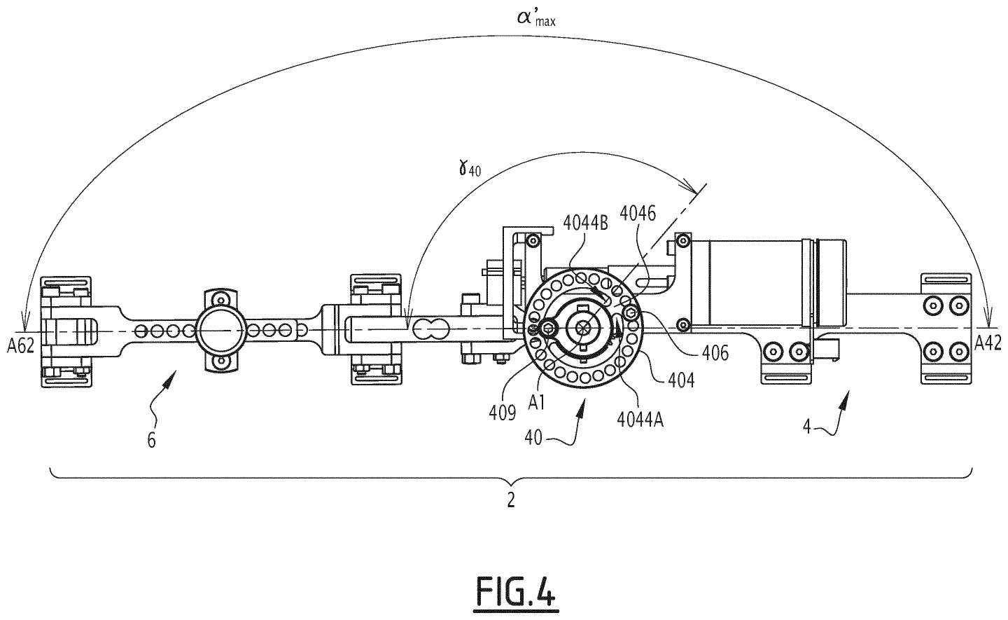

FIG. 4 is a side view similar to FIG. 3 when the orthosis is in an extended position;

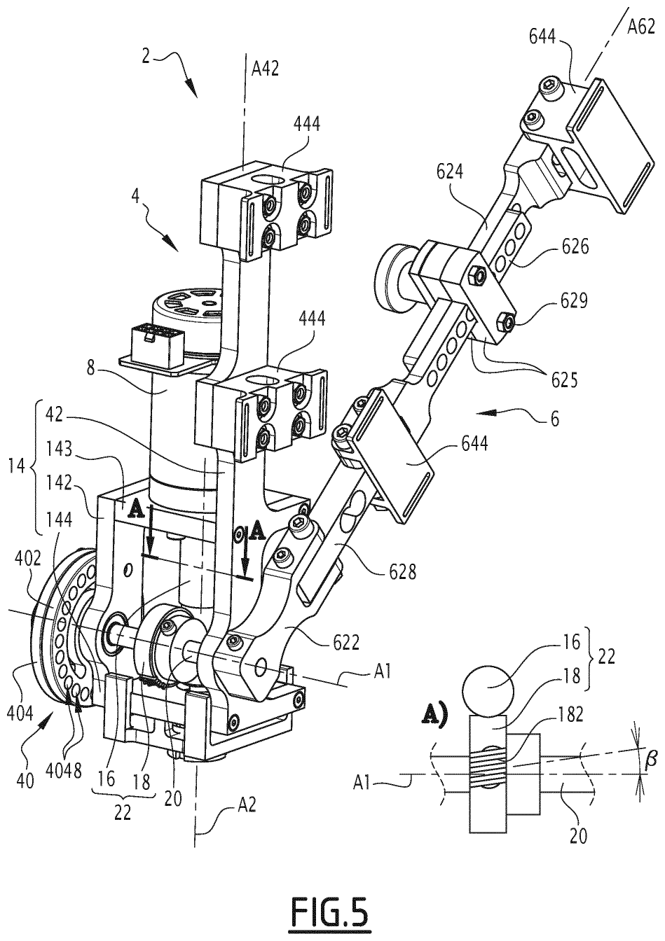

FIG. 5 is a perspective view of the orthosis in the configuration and position of FIG. 3, insert A corresponding to a partial cut view in the direction of arrows A-A on FIG. 5;

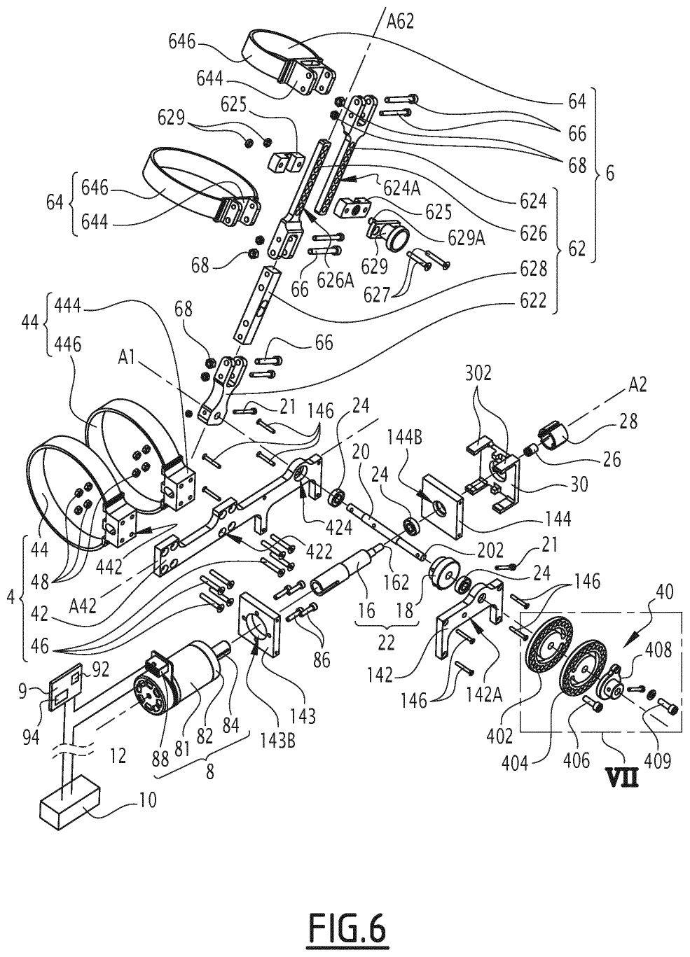

FIG. 6 is an exploded perspective view of the orthosis of FIGS. 1 to 5, seen from an angle different from the one of FIG. 5;

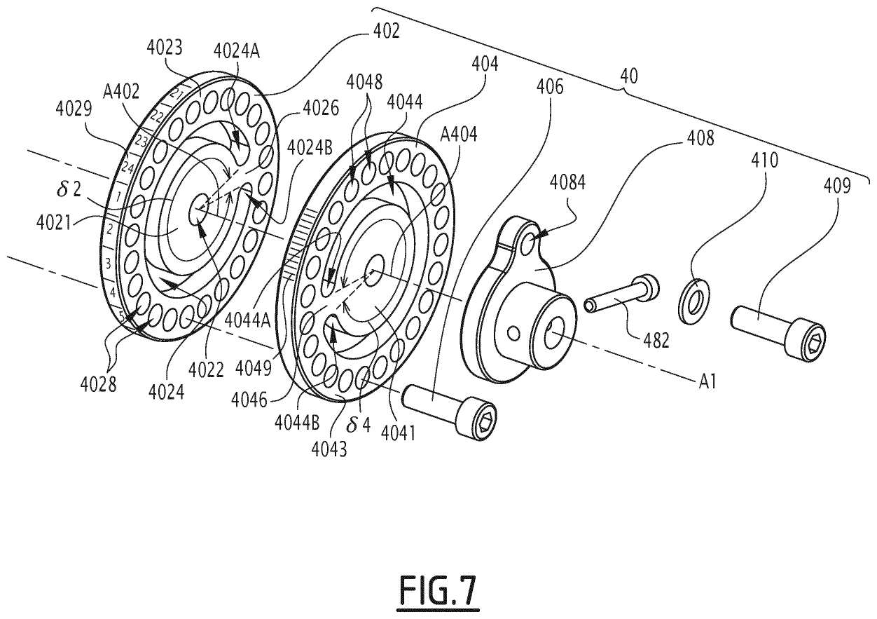

FIG. 7 is an enlarged perspective view of detail VII on FIG. 6 and

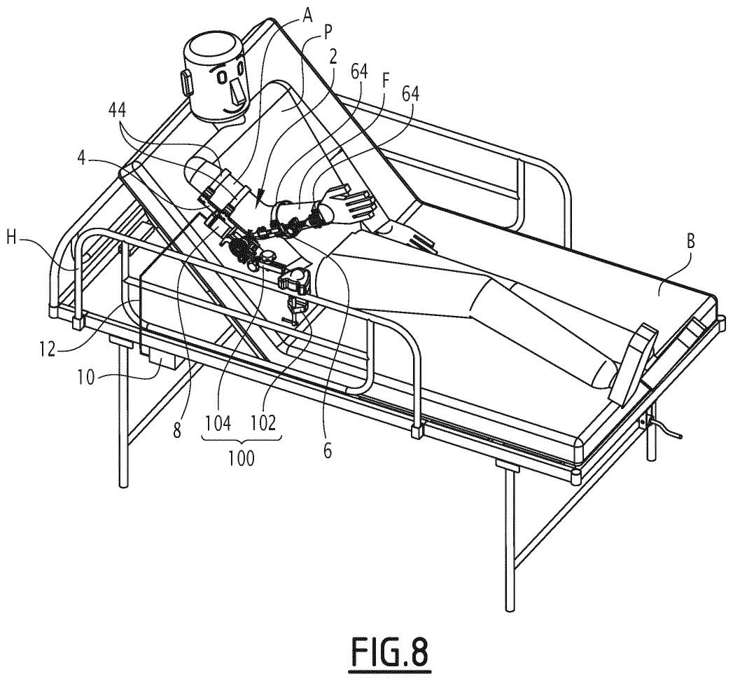

FIG. 8 is a representation of the use of the orthosis of FIGS. 1 to 7 on the right arm of a patient laying on a medical bed.

The elbow orthosis 2 represented on FIGS. 1 to 8 includes a first fitting 4 configured for being mounted on an arm A of a patient P and a second fitting 6 configured for being mounted on the forearm F of the patient who, in the example of FIG. 8, is laying on a medical bed B.

Alternatively, the patient may be seated on a chair or stand.

The first fitting 4 includes an armature first 42, made of a metallic plate, and two braces 44. A42 denotes a longitudinal axis of the armature 42. Each brace 44 is immobilized on the armature 42 by four screws 46 cooperating with four nuts 48. Each screw 46 crosses two through holes 422 and 442 respectively provided in the armature 42 and in a base portion 444 of a brace 44.

The second fitting 6 includes another armature 62 and two braces 64. A62 denotes a longitudinal axis of the armature 62. The armature 62 includes a rigid elongated bracket 622 and two perforated rods 624 and 626 movable with respect to each other along the axis A62, in order to adapt the length of the second fitting 6 to the actual length of the patient's forearm. Each rod 624 or 626 is provided with a series of through holes 624A, respectively 626A. The rods 624 and 626 are guided with respect to each other by two clamps 625 held in position by two screws 627 cooperating with corresponding nuts 627A. A knob 629 is mounted on the clamps 625 and provided with a pin 629A adapted to penetrate in a group of two through holes 624A and 626A aligned along a direction perpendicular to the longitudinal axis A62, so as to fix the length of the sub-armature made of the rods 624 and 626.

A strain gauge 628 is inserted, along the longitudinal axis A62, between the bracket 622 and the sub-armature made of the rods 624 and 626. This strain gauge 628 measures the deflection of the armature 62 and translates it into torque at the level of an axis A1, which is a first rotation axis between the fittings 4 and 6. This allows detecting a torque applied, between the two fittings 4 and 6, on an articulation mechanism of the elbow orthosis 2.

A base portion 644 of each brace 64 is mounted at one end of a rod 624 or 626 by respective screws 66 cooperating with corresponding nuts 68. A pair of screws 66 is also used for fastening the strain gauge 628 to one of the rods, rod 626 in the example of the figures. Another pair of screws 66 is used for fastening the strain gauge 628 to the bracket 622.

446 and 646 respectively denote a flexible strap of a brace 44 or 64. For the sake of simplicity, the straps 446 and 646 are represented on FIGS. 6 and 8 only.

The two fittings 4 and 6 are articulated around the first rotation axis A1.

The two longitudinal axes A42 and A62 are respectively radial with respect to the first rotation axis A1.

α denotes an angle measured between the two longitudinal axes A42 and A62 above the first rotation axis A1 in the right arm configuration represented on FIGS. 1 and 2. This angle α can vary between a minimum value αₘᵢₙ corresponding to the flexed position, or folded position, of FIG. 1 and a maximum value αₘₐₓ corresponding to the extended position of FIG. 2. It should be noted here that the maximum value αₘₐₓ can be strictly greater than 180°, as shown in FIG. 2, which corresponds to some morphologies.

The minimum and maximum values for angle α when the orthosis 2 is configured for the left arm of a given patient can be equal to or different from the corresponding values in the configuration for a right arm, as shown respectively by values α′ₘᵢₙ and α′ₘₐₓ on FIGS. 3 and 4. Here value α′ₘᵢₙ equals value α′ₘᵢₙ and value α′ₘₐₓ is different from value αₘₐₓ.

An electric gear-motor 8 also belongs to the orthosis 2 and includes a brushless DC motor 81, a reducer 82 and an output shaft 84 aligned on a second rotation axis A2 perpendicular to, and non-secant with, the first rotation axis A1.

In the example of the figures, axes A2 and A42 are parallel. However, another relative orientation is possible.

Advantageously, the reducer 82 is an epicyclical reducer. However, another type of reducer can also be used.

The gear-motor 8 is capable of delivering a torque large enough to move the second fitting 6 with respect to the first fitting 4, in a flexion or extension movement of the elbow around the first rotation axis A1, even with no help of the patient, for instance when the spasticity of the patient's muscles prevents him or her to assist this movement. This is essential in order to help a patient to move his or her arm, even if he cannot do it with his or her own muscles, for instance in the few days after a cardio-vascular accident.

An electronic control unit or ECU 9, in the form of a printed circuit board, is coupled to the motor 81 and includes a microcontroller 92, a memory 94 and non-represented other electronic components, as needed. The electronic control unit is configured for piloting the electric gear-motor 8 and for supplying it with electric power coming from a battery set 10 via an electric line 12. For the sake of simplicity, items 9, 10 and 12 are represented on FIGS. 6 and 8 only. The ECU 9 and the electric line 12 are connected to a connector 88 of the gear-motor 8.

Alternatively, in a non-represented alternative embodiment of the invention, instead of a battery set, one can use a mains supply for supplying the gear-motor with electric power.

A box-like structure 14 is made by four flat armatures cut in metallic flat panels, for instance made of aluminum or iron. In the example of the figures, the four armatures of the box-like structure 14 have the same thickness and are cut in the same aluminum panel. Alternatively, they can have different thicknesses.

The first armature of the box-like structure is formed by the armature 42. A second armature 142 of the box-like structure is formed by a C-shaped plate, which extends parallel to the first armature 42 in the mounted configuration of box-like structure 14. A third armature 143 and a fourth armature 144 of the box-like structure are made with square plates and extend between the first and second armatures 42, 142, perpendicular to these first and second armatures and to the second rotation axis A2. The third and fourth armatures 143 and 144 are parallel. The box-like structure has a globally parallelepiped shape. Each third or fourth armature 143 or 144 has a central circular through hole 143B, respectively 144B, aligned on the second rotation axis A2 in the mounted configuration of box-like structure 14 and of the orthosis 2.

The armatures 42, 142, 143 and 144 of the box-like structure 14 are held together by screws 146. On the other hand, the electric gear-motor 8 is mounted on the third armature 144 and immobilized thereon by four screws 86.

A threaded rod 16 is secured in rotation to the free end of the output shaft 84 and extends within the box-like structure 14, along the second rotation axis A2. This threaded rod 16 cooperates with a toothed wheel 18 mounted on, and secured in rotation with, a rotation shaft 20, which extends along the first rotation axis A1.

The connection in rotation around the first rotation axis A1, between the toothed wheel 18 and the rotation shaft 20, is made via a screw 21 inserted in two aligned housings respectively provided in the toothed wheel and in the rotation shaft. Alternatively, a key or a retractable pin can be used instead of the connection screw 21. In such a case, the key or pin is preferably force-fitted in the wheel 18 and/or in the shaft 20.

Parts 16 and 18 together form a worm-and-wheel transmission 22.

β denotes an inclination angle between the teeth 182 of the toothed wheel 18 and the first rotation axis A1. This angle is visible on insert A) of FIG. 5 and chosen so that the worm-and-wheel transmission 22 is reversible. Due to the reversibility of the worm-and-wheel transmission, the threaded rod 16 can drive the toothed wheel 18 in a normal operation mode of the orthosis 2 and the toothed wheel can drive the threaded rod in a setting mode of the elbow orthosis, as explained here below. In practice, the angle β is chosen above 10°

The rotation shaft 20 is supported within the box-like structure 14, namely between the first and second armatures 42 and 142, by two bearings 24 respectively mounted on the first and second plates.

The rotation shaft 20 crosses the first and second armatures 42 and 142. More precisely, the rotation shaft 20 crosses a through hole 424 of the first armature 42 and is secured to the extremity of the bracket 622 opposed to the strain gauge 628 via another screw 21, in such a way that the rotation shaft 20 and the other armature 62 rotate together around the first rotation axis A1. In other words, the bracket 622 of the second fitting 6 is secured to the rotation shaft 20 via the other screw 21. Alternatively, a key or a retractable pin can be used instead of the other connection screw 21. In such a case, the key or pin is preferably force-fitted in the wheel 18 and/or in the shaft 20.

A third bearing 24 supports the threaded rod 16 within the bore 144B. A cylindrical magnet 26 is mounted at the free end 162 of the threaded rod opposite to the output shaft 84 and secured on the threaded rod 16 in rotation around the second rotation axis A2.

Advantageously, the magnet 26 is glued onto the free end 162.

With the articulation mechanism made of sub-assemblies 14 and 22 and of the rotation shaft 20, it is possible to drive the second fitting 6 in rotation around the first rotation axis A1 by actuating the electric motor 8, so as to rotate the threaded rod 16, in one direction or the other, around the second rotation axis A2, thus the second fitting 6 around the first rotation axis.

An absolute rotation sensor 28 is mounted on a support member 30 attached to the fourth armature 144. With this respect, the support member 30 is provided with four flexible legs 302 hooked onto the fourth armature 144.

The rotation sensor 28 is mounted on the support member 30.

The absolute rotation sensor 28 allows detecting a rotation of the toothed rod 16 around the second rotation axis A2, thus to detect a rotation of the other armature 62 around the first rotation axis A1 with respect to the first armature 42, assuming that the worm-and-wheel transmission has a negligible play.

On the other hand, the strain gauge 628 allows detecting a torque applied on the articulation mechanism made of parts 14 and 22.

The output signal of the rotation sensor 28 is transferred to the electronic control unit 9 via a non-represented data line. Similarly, the output signal of the strain gauge 628 is transferred to the electronic control unit 9 via another non-represented data line.

The limits of the angular displacement of the second fitting 6 with respect to the first fitting 8 can be set electronically in the memory of the electronic control unit 9, via proper programming of this electronic control unit.

For the sake of safety, a mechanical stop arrangement 40 is used for limiting the angular movement of the second fitting 6 with respect to the first fitting 4, in particular in case of improper programming of the electronic control unit 9 or in case of a defective rotation sensor 28.

This mechanical stop arrangement 40 is supported by the box-like structure 14 and includes a first circular plate 402 and a second circular plate 404.

In the example of the figures, the two plates 402 and 404 are identical, which is advantageous in terms of manufacturing and maintenance. However, this is not compulsory.

The first plate 402 includes a first central bore 4022 centered on a central axis A402 of the first plate 402 and adapted to accommodate a portion of the rotation shaft 20. The first plate 402 includes first circular slot 4024 centered on the central axis A402. Similarly, the second plate 404 includes a central bore 4042, centered on a central axis A404 of the second plate 404 and adapted to accommodate a portion of the rotation shaft 20, and a circular slot 4044 in the form of an arc of a circle centered on the axis A404.

In mounted configuration of the orthosis 2, axes A1, A402 and A404 are superimposed and define a common central axis for the rotation shaft 20 and for the first and second plates 402 and 404. The two plates 402 and 404 are mounted next to each other along this common axis and the rotation shaft 20 crosses the central bores 4022 and 4042.

Each circular slot 4024 or 4044 extends around the corresponding central axis A402 or A404 on an angular sector whose apex angle δ₂, respectively δ₄, is strictly less than 360°, in practice less than 330°, so that a bridge 4026 or 4046 connects a central portion 4021 or 4041 of the plate 402 or 404, where the central bore 4022 or 4042 is located, to a peripheral portion 4023 or 4043 of the same plate.

Thus, the adjective “circular” used for qualifying the slots 4024 and 4044 does not means that they are annular, but that they extend each on an arc of a circle.

Here, angles δ₂ and δ₄ are identical since the first and second plates 402 and 404 are identical. However, this is not compulsory.

4024A and 4024B respectively denote the two extremities of the circular slot 4024 located on either side of the bridge 4026. Similarly, 4044A and 4044B respectively denote the two extremities of the circular slot 4044 located on either side of the bridge 4046.

When the first and second plates 402 and 404 are mounted on the rotation shaft 20, depending on their respective orientation around the axis A2, that is depending on the position of the bridges 4026 and 4046, a greater or lesser portion of the circular slots 4024 and 4044 overlap.

A circular series of twenty-four through holes 4028 or 4048 is drilled in each peripheral portion 4023 or 4043. Each series of twenty-four through holes 4028 or 4048 is centered of the central axis A402 or A404 of the corresponding plate 402 or 404.

The relative orientation of the first and second plates 402 and 404 around the first rotation axis A1, thus around the common axis, is set by rotating each plate 402 and 404 around its central axis A402, A404 aligned on the first rotation axis A1. Once the relative orientation of the first and second plates 402 and 404 is set, that is once the bridges 4026 and 4046 are correctly positioned, this orientation can be fixed by inserting a screw 406 into two aligned through holes 4028 and 4048 and by screwing this screw in a threaded hole 142A of the second armature 142. This setting of the relative orientation of the first and second plates 402 and 404 belongs to the action of configuring the elbow orthosis 2.

S4A denotes a first combined slot made through the first and second plates 402 and 404 by two overlapping portions of the circular slots 4024 and 4044. S4B denotes a second combined slot made through the first and second plates 402 and 404 by two other overlapping portions of the circular slots 4024 and 4044, on the opposite side of the bridges 4026 and 4046.

On the other hand, a crank 408 is secured at the end 202 of the rotation shaft 20 opposite to the bracket 622, via an additional screw 482. Alternatively, a key or a retractable pin can be used instead of the connection screw 482. In such a case, the key or pin is preferably force-fitted in the wheel 18 and/or in the shaft 20.

A pin 409 is mounted on the crank 408 with interposition of a washer 410 and crosses a through hole 4084 provided on the crank. When the crank 408 is mounted on the rotation shaft 20, the pin 409 extends parallel to the first rotation axis A1. This pin 409 penetrates into one of the combined slots S4A or S4B. Let's assume the pin 409 penetrates into the combined slot S4A, as shown on FIGS. 1 and 2.

The angular movement of the pin 409 is limited by the stops formed by the extremities 4024A, 4024B, 4044A or 4044B of the overlapping portions of the circular slots 4024 and 4044. In practice, these extremities are defined by the side edges of the bridges 4026 and 4046.

As can be deduced by the comparison of FIGS. 1 and 2, once the screw 406 has been screwed within the threaded hole 142A through the two aligned through holes 4028 and 4048 and once the pin has been introduced into the combined slot S4A, it can slide within the combined slot S4A when following the rotational movement of the rotation shaft, to which it is secured in rotation via the crank 408 and the screw 482.

The combined slot S4A extends on an arc of the circle having an apex angle Y40 defined between the bridges 4026 and 4046, more precisely between the extremities 4024A and 4044A. This angle γ₄₀ is the amplitude of the maximum angular movement of the second fitting (6) with respect to the first fitting (4), around the first rotation axis (A1).

This angle γ₄₀ is equal to the difference between the maximum and minimum angles αₘₐₓ and αₘᵢₙ mentioned here-above.

Therefore, the following equation prevails:

$${γ}_{40} = {a}_{max} - {a}_{min} \qquad ( equation 1 )$$

A proper definition of the orientation of the two plates 402 and 404 around the first rotation axis A1 allows setting the value of the angle γ₄₀, thus of the maximum amplitude of the relative movement between the first and second fittings 4 and 6. The simple structure of the mechanical stop arrangement makes it easy and intuitive to use for setting this angle γ₄₀.

The mechanical stop arrangement 40 is a safety guard and its configuration, that is the action of setting the angle γ₄₀, can be made in conjunction with the programming of the ECU 9, where control parameters can be set for limiting the movements of the output shaft 84. Thus, the mechanical stop arrangement 40 is used mainly in case of a default in the programming of the ECU 9 or a defective component of this ECU.

In addition, the simple and intuitive structure of the mechanical stop arrangement 40 allows quickly switching between the right arm configuration represented on FIGS. 1 and 2 and the left arm configuration represented on FIGS. 3 and 4, by changing the relative position of the first and second plates 402 and 404 around the first rotation axis A1 and the control parameters of the gear-motor 8 stored in the memory of the ECU 9.

In particular, it is possible to switch between a configuration of the elbow orthosis for a right arm and a configuration of the elbow orthosis for a left arm by inverting its most extended position and its most flexed position defined by the combined slots S4A and/or S4B, that is by the overlapping portions of the first and second circular slots 4024 and 4044.

According to an advantageous aspect of the invention, the reversibility of the worm-and-wheel transmission 22 can be used upon configuration of the elbow orthosis 2.

According to a first approach, once the mechanical stop arrangement 40 has been set the elbow orthosis 2 can be configured by moving the second fitting 6 with respect to the first fitting 4, in order to align the first fitting on the arm A of the patient P and the second fitting 6 on the forearm F of the patient, when the gear-motor 8 is not energized. This allows adjusting the opening angle of the elbow orthosis to the actual rest position of the patient's arm and forearm. Thereafter, the first and second fitting can be respectively secured to the patient's arm and forearm.

According to a second approach, still using the reversibility of the worm-and-wheel transmission 22 and once the mechanical stop arrangement 40 has been set and the braces 44 and 64 have been closed around the arm A and forearm F of the patient P, it is possible for a medical practitioner to move the forearm of a patient around the first articulation axis, when the gear-motor 8 is not energized, so as to bring the pin 409 into the extremities of the combined slot S4A, in contact with the bridges 4026 and 4046, while taking care of not hurting the patient. This allows checking that the two limit positions, defined by the ends of the combined slot S4A where the pin 409 is engaged, are compatible with the morphology and pathology of the patient and that the elbow orthosis 2 will not hurt the patient when the gear-motor is actuated.

According to an optional aspect of the invention represented on FIG. 7 only, a graduation mark 4029 is provided on the outer edge of the first plate 402, here in the form of twenty-four numbers identifying each through hole 4028 located nearby. This allows identifying the angular position of the first plate with respect to the screw 406 or a non-represented mark provided on the second armature 142.

In a non-represented alternative embodiment of the invention, the graduation mark 4029 includes angular indications, instead of twenty-four numbers.

According to a non-represented variant, a graduation mark can also be provided on the outer edge of the second plate 404, also here in the form of twenty-four numbers. Then the relative orientation of the two plates 402 and 404 around the first rotation axis A1 can be identified by a combination of two numbers or of angular indications visible on their edges, near the screw 406.

According to another optional aspect of the invention represented on FIG. 7 only, a knurled zone 4049 is provided on the outer edge of second plate 404. This knurled zone facilitates the application of a torque on the second plate, around its central axis A404, during setting of the configuration of the mechanical stop arrangement 40.

As shown on FIG. 7, the knurled zone 4049 extends on a limited angular sector of the outer edge of the second plate 404. In a variant, the knurled zone 4049 extends overall this edge, on 360°.

According to a non-represented variant, a knurled zone can also be provided on the outer edge of the first plate 402.

The graduation mark 4029 and the knurled zone 4409 can also be combined.

In a non-represented alternative embodiment of the invention, the graduation mark 4029 and/or he knurled zone 4049 is located outside the outer edge of the peripheral portion 4023 and/or 4043.

As represented on FIG. 8, a support member 100 is provided of supporting the elbow orthosis 2 from a handrail H of the medical bed B. This avoids the patient having to bear the weight of the orthosis. This support member 100 includes an anchoring part 102 configured for being locked on the handrail H and a bracket 104 articulated on the anchoring part 102 and provided with a clamp for holding a part of the orthosis, for instance the box-like structure 14. This allows the support member 100 to support at least items 4, 6, 8, 14, 20 and 22.

The support member 100 is adapted to the environment of the patient P. For instance, if the patient is seated in a wheelchair, the anchoring part 102 is configured for being fixed on an armrest of the wheelchair.

The battery set 10 is also mounted on the medical bed B and connected to the gear-motor via the connection line 12 formed of a bundle of electric wires, which relieves the patient of supporting the weight of this battery set. However, alternatively, the battery set 10 can be supported by one of the first and second fittings 4 and 6.

If a mains supply is used, as considered here above, no battery set needs to be supported and the electrical line might be plugged on a wall socket or on a current converter.

The embodiment, variants and optional aspects listed here-above may be combined to generate other embodiments of the invention defined in the appended claims.

1. An elbow orthosis (2) comprising a first fitting (4) configured to be mounted on an arm (A) of a patient (P), a second fitting (6) configured for being mounted on a forearm (F) of the patient, an articulation mechanism (14, 20, 22) between the first and second fittings and an electric gear-motor (8) mounted on the first fitting for moving the second fitting with respect to the first fitting in rotation around a first rotation axis (A1) of the articulation mechanism, wherein
an output shaft (84) of the electric gear-motor is aligned on a second rotation axis (A2) perpendicular to the first rotation axis and
the orthosis is equipped with
an angular sensor (28) for sensing the angular position of the second fitting (6) with respect to the first fitting (4) around the first rotation axis (A1) and
a torque sensor (628) for sensing a torque exerted on the articulation (14, 22) mechanism by the electric gear-motor (8);
a worm-and-wheel transmission (22) drives a rotation shaft (20), which defines the first rotation axis, from the rotational movement of the output shaft (84) of the electric gear-motor;
the second fitting (6) is secured to the rotation shaft; and
a mechanical stop arrangement (40) is configured for limiting the angular movement of the second fitting (6) with respect to the first fitting (4) around the first rotation axis (A1),
the mechanical stop arrangement (40) includes:
a first plate (402) provided with a first circular slot (4024) and a first series of through holes (4028) spread on a circle centered on a central axis (A402) of the first circular slot;
a second plate (404) provided with a second circular slot (4044) and a second series of through holes (4048) spread on a circle centered on a central axis (A404) of the second circular slot; and
a crank (408) secured to the rotation shaft (20) and equipped with a pin (409) engaged in the first and second circular slots (4024, 4044);
the first and second plates (402, 404) are aligned on a common central axis (A1, A402, A404) and their relative orientation around this common axis is set by aligning at least one through hole (4028) of the first series with one through hole (4048) of the second series;
some portions of the first and second circular slots (4024, 4044) overlap, depending on the orientation of the first and second plates around the common axis; and
the respective extremities (4024A, 4024B, 4044A, 4044B) of the overlapping portions (S4A, S4B) of the first and second circular slots (4024, 4044) define the amplitude (γ₄₀) of the maximum angular movement of the second fitting (6) with respect to the first fitting (4), around the first rotation axis (A1), when it is driven by the worm-and-wheel transmission (22).

2. The elbow orthosis of claim 1, wherein the worm-and-wheel transmission (22) is reversible, in such a way that a threaded rod (16) of the worm-and-wheel transmission can drive a toothed wheel (18) of the worm-and-wheel transmission in a normal operation mode of the orthosis (2) and the toothed wheel can drive the threaded rod in a setting mode of the orthosis.

3. The elbow orthosis of claim 2, wherein an inclination angle (B) between the teeth (182) of the toothed wheel (18) and the first rotation axis (A1) is above 10°.

4. The elbow orthosis of claim 1, wherein it is configurable for use on a right or left arm, by changing the relative angular position of the first plate (402) with respect to the second plate (404) and some control parameters of the electric gear-motor (8).

5. The elbow orthosis of claim 1, wherein it includes a box-like structure (14) for supporting the gear-motor (8), the worm-and-wheel transmission (22), the rotation shaft (20) and the mechanical stop arrangement (40).

6. The elbow orthosis of claim 5, wherein the box-like structure (14) includes four armatures (42, 142, 143, 144) assembled together by screws (146), wherein the worm-and-wheel transmission (22) is mounted within the box-like structure, wherein the wheel (18) of the worm-and-wheel transmission is mounted on the rotation shaft (20) between two parallel armatures (42, 142) of the box-like structure and wherein the rotation shaft (20) is mounted on these two parallel armatures with a possibility of rotation.

7. The elbow orthosis of claim 1, wherein at least one of the first and second plates (402, 404) includes a graduation mark (4029) for identifying the relative orientation of the first and second plates.

8. The elbow orthosis of claim 7, wherein the graduation mark (4029) is formed on an edge of the at least one plate (402, 404).

9. The elbow orthosis of claim 1, wherein the first and/or second plate (402, 404) is provided with a knurled zone (4049).

10. The elbow orthosis of claim 1, wherein it includes a support member (100) for supporting at least the first and second fittings (4, 6), the electric gear-motor (8), the articulation mechanism (14, 22), the angular sensor (28), the torque sensor (628) and the worm-and-wheel transmission (22) with respect to a structure (H).

11. The elbow orthosis of claim 1, wherein it includes a battery set (10) for providing electric power to the electric gear-motor (8) and to an electronic control unit (9) for controlling the electric gear-motor and wherein the battery set is either supported by one of the first and second fittings (4, 6) or located in the vicinity of the first and second fittings and connected to the electric gear-motor via a bundle of electrical wires (12).

12. A process for adapting a configuration of an elbow orthosis (2) according to claim 1 to the morphology and/or pathology of a patient (P), this method including at least the following steps:
a) adjusting the respective angular position of the first and second plates (402, 404) around their common central axis (A1, A402, A404), so as to define with their overlapping first and second circular slots a path (S4A, S4B) for the pin (409) mounted on the crank (408) secured to the rotation shaft (20); and
b) immobilizing the first and second plates in their respective angular positions by insertion of a locking member (406) in one through hole (4028) of the first series and in one through hole (4048) of the second series.

13. The process of claim 12, wherein the worm-and-wheel transmission (22) in the elbow orthosis is reversible, in such a way that a threaded rod (16) of the worm-and-wheel transmission can drive a toothed wheel (18) of the worm-and-wheel transmission in a normal operation mode of the orthosis (2) and the toothed wheel can drive the threaded rod in a setting mode of the orthosis, and the process includes a further step consisting in:
c) moving the second fitting (6) with respect to the first fitting (4), in rotation around the first rotation axis (A1), in order to align the first fitting on the arm (A) of the patient (P) and the second fitting on the forearm (F) of the patient when the electric gear-motor is not energized, using the reversibility of the worm-and-wheel transmission; and
d) securing the first fitting (4) to the arm of the patient and the second fitting (6) to the forearm of the patient.

14. The process of claim 12, wherein the elbow orthosis is configurable for use on a right or left arm, by changing the relative angular position of the first plate (402) with respect to the second plate (404) and some control parameters of the electric gear-motor (8) and wherein the passage of a configuration for a left arm to a configuration for a right arm, or vice versa, occurs by inverting the most extended position and the most flexed position of the orthosis defined by the overlapping portions (S4A, S4B) of the first and second circulars slots (4024, 4044).

15. The process of claim 12, wherein it includes a supplementary step implemented after b) and consisting in:
e) moving the second fitting (6) with respect to the first fitting (4) around the first rotation axis (A1), while the electric gear-motor (8) is not energized, up to bringing the pin (409) into the respective extremities (4024A, 4024B, 4044A, 4044B) of the overlapping portion (S4A, S4B) of the first and second circular slots (4024, 4044), in order to assess the maximum relative angular movement of the first and second fitting around the first rotation axis.
